(19) United States
(12) Patent Application Publication
(10) Pub. No.: US 2006/0144426 A1
(43) Pub. Date: Jul. 6, 2006

(54) Towelette, and method

(76) Inventor: Arlene Mathews, Overland Park, KS (US)

Correspondence Address: DAVID E. HERRON II, PO BOX 2778, KANSAS CITY, KS, 66110, US

(21) Appl. No.: 11/236,773

(22) Filed: Sep. 28, 2005

Related U.S. Application Data
(60) Provisional application No. 60/640,468, filed on Dec. 31, 2004.

(51) Int. Cl. A47K 7/03; A61L 2/16

(52) U.S. Cl. 134/42; 15/209.1; 510/438; 422/28

(57) ABSTRACT

The invention is a towelette, as well as a sanitation method that incorporates use of the inventive towelette.

The invention is a cleansing towelette, a method for using an inventive towelette, and well as a sanitation method.

### THE INVENTIVE METHOD

The inventive method includes the steps of imprinting a message upon a towelette and impregnating the towelette with a cleaning agent. The method will also include the step of concealing the message, then rubbing a selected area of skin with the towelette, thereby cleansing the selected area and enabling the message to become apparent.

Optionally, the inventive method may also include the steps of running water over the selected area. The inventive method may also include the step of rubbing the selected area while adjacent a water faucet having at least one knob, and positioning the towelette over the at least one knob, then turning the at least one knob to cease flow of the running water.

In a preferred embodiment, the inventive method may include the step of imprinting the message using a substantially clear oleophilic compound, such as a terpene or sterol, upon the towelette. Specifically, the oleophilic compound may comprise camphor oil.

In a second preferred embodiment, the inventive method may include the step of applying a powdery soap to the towelette, thereby covering the message. Optionally, the message may be generally positioned in a central location on the towelette, and a heavier concentration of the powdery soap applied to the area of the towelette including the message. In this way, the message can be substantially concealed on the towelette beneath the powdery soap. In this embodiment, as a user scrubs a selected area of skin using the towelette, the powdery soap-like coating will erode from the towelette, leaving the message exposed and available for viewing unapparent to a viewer.

In another preferred embodiment, the inventive method may also include the step of moistening the towelette, and sprinkling a powdery soap over the towelette. The inventive method may also include the step of drying the towelette, such as by air pressure or heating the towelette.

The inventive method may include a variety of products satisfactory to serve as a cleaning agent. For example, the cleaning agent may include a cholorhexidine alcohol solution, or any well-known antimicrobial or antibacterial soap.

### THE INVENTIVE TOWELETTE

The invention also includes an inventive towelette that includes a porous cloth like material, and a cleaning agent impregnating the material. The inventive towelette will also include a message included within the material. As one rubs a selected area of skin with the material, the area is cleansed and the message converts from concealed to visible.

In a preferred embodiment of the towelette, the invention will include a substantially clear oleophilic compound imprinted into a message upon the towelette. As the area is cleansed, the oleophilic compound gathers dirt, oils, grime, and other material, thereby revealing the message using the oleophilic compound.

The oleophilic compound may be selected as a terpene or a sterile; a preferred terpene is camphor oil. Of course, any well-known oleophilic compound would be suitable.

In a preferred embodiment of the invention, the towelette may include a cleaning agent comprising an opaquing material that at least partly conceals the message. In this embodiment, as the area of skin is cleansed, the cleaning agent erodes from the towelette and reveals the message. Alternatively, the message is imprinted at a central location on the towelette and the cleaning agent generally includes an opaque soap that given greater concentration in the generally central location. The greater concentration of cleaning agent substantially obscures the message until the cleaning agent is removed by a hand washing procedure using the towelette.

In another preferred embodiment of the invention, the message is imprinted onto the towelette using a solution that alters its color when it becomes wet. For example, numerous ionic salts, such as cobalt chloride turn color when exposed to water. A solution containing a low concentration of an ionic salt, such as cobalt chloride, may be imprinted or etched onto the material.

Other objects, advantages, and novel features of the present invention will become apparent from the following detailed description of the invention when considered in conjunction with the accompanying drawings.

### BRIEF DESCRIPTION OF THE DRAWINGS

FIG. 1 is a plan view showing a towelette, according to the principals of the invention, wherein the towelette is in its pre-use condition.

FIG. 2 is a plan view of the towelette, shown at an intermediate phase wherein the message is becoming apparent.

FIG. 3 is a plan view of the inventive towelette, shown post-use with the message readily apparent.

FIG. 4 shows a perspective view of the towelette in combination with a dispenser.

FIG. 5 is a plan view showing the towelette how it would be commonly used.

FIG. 6 shows a perspective view showing the towelette in use.

FIG. 7 is a perspective view showing the towelette being discarded after use.

### DETAILED DESCRIPTION OF THE PREFERRED EMBODIMENTS

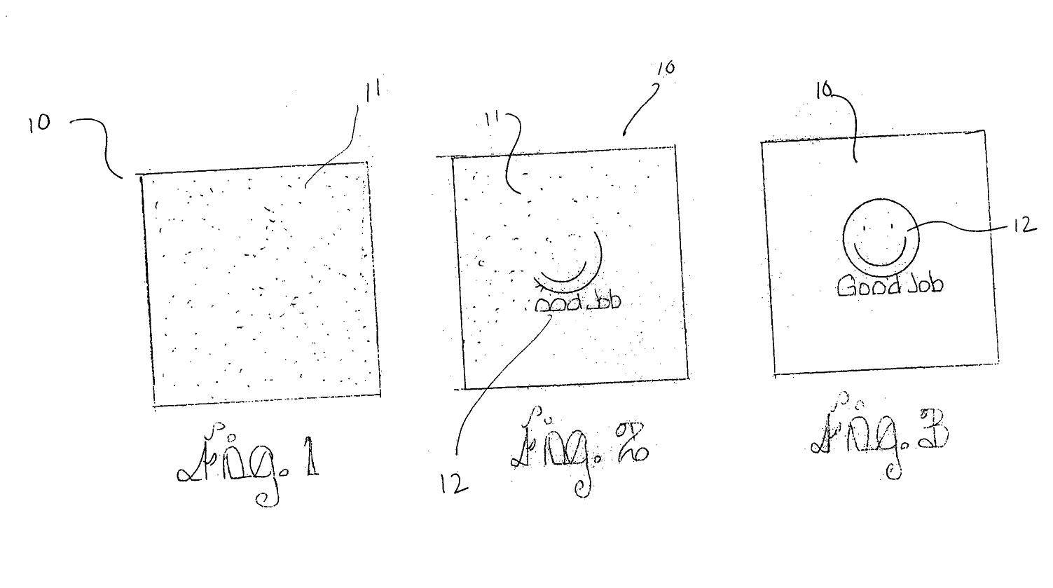

FIG. 1 shows a plan view of the inventive towelette 10 in its pre-used condition. The surface of the towelette 10 is covered with a cleaning agent 11. As shown in FIG. 1, the cleaning agent 11 substantially covers the entire surface of the towelette, and no message is viewable before the towelette 10 is used in a sanitation procedure.

FIG. 2 shows a plan view of the inventive towelette 10 having the cleaning agent 11 covering at least a portion of the surface of the towelette 10. As the towelette is used in a cleansing procedure, a message 12 begins to become viewable. The message 12 may be imprinted on to the towelette by any number of methods. For example, the message 12 may be imprinted onto the towelette 10 by using a substantially clear oleophilic compound that attracts, grabs, and retains dirt and oils during a cleansing procedure, thereby making the message 12 become more apparent. Alternatively, the message 12 maybe imprinted onto the towelette 10 by using a colored ink or dye that can be substantially covered and opaqued by cleaning agent 11.

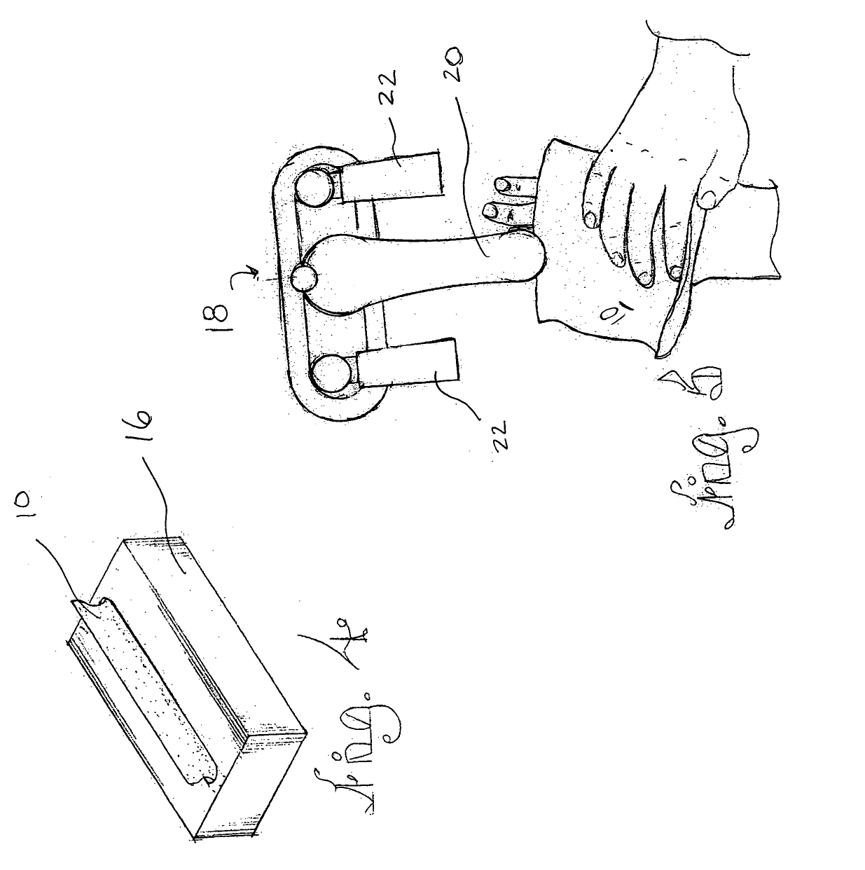

FIG. 3 is a plan view of the inventive towelette 10, shown in a post-use condition whereby message 12 is readily and completely apparent. It should also be noted that message 12 may be etched or imprinted onto the towelette 10 by using a solution of an ionic salt, such as cobalt chloride, which is known to change color when exposed to water. Alternatively, an oleophilic compound, such as a terpene, sterol oil, such as camphor oil or the like, can be used to etch the message 12 onto the towelette 10.

Still referring to FIG. 3, message 12 includes a “happy face” and a short message, such as “good job”. Of course, other indicators or messages are possible such as children's characters, or the like. In another embodiment of the method, the message 12 can be any number of different characters or cartoon icons. In this way, the towelette 10 may encourage children to wash well in order to view an image of their favorite cartoon character, and said image would not be viewable until the child thoroughly scrubs. In a preferred embodiment, the towelette is dispensed as a dry towelette. Alternatively, the towelette may be moistened with a fluid, or an alcohol based product.

FIG. 4 shows a perspective view of towelette 10 in combination with a dispenser 16. The embodiment, shown in FIG. 4 works best with a dry towelette 10, or only a slightly moist version thereof. The dispenser 16 commonly allows a user to grab one towelette 10 at a time, of course, other dispensing methods, such as a roll or a stacking means, are also possible and within the scope of the principles of this invention.

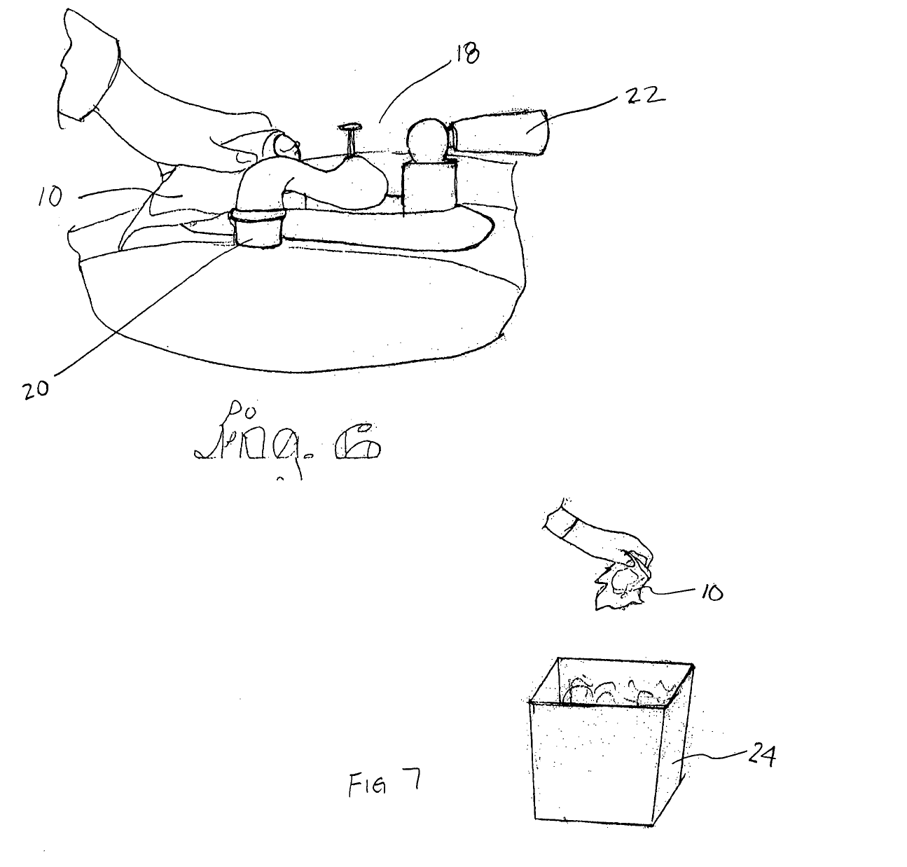

FIG. 5 shows the inventive towelette 10 in use adjacent a water faucet 18 the faucet includes a spigot 20, and at least one knob 22. As the user washes his hands beneath flowing water from the spigot 20, the towelette 10 is wrung and wiped vigorously across a selected area of skin. During the course of this procedure, the message (as seen in FIGS. 2, 3) gradually becomes more readily apparent. As the procedure as shown in FIG. 5, becomes more complete, the message 12 becomes completely apparent (see FIG. 3).

FIG. 6 is a perspective view showing a final step of a hand washing procedure. As one completes the hand washing procedure, the used towelette 10 is placed over a knob 22, then the knob is turned to shut off the flow of water from the spigot 20. Using the used towelette 10 to grip the knob 22 assists in preventing spread of germs or bacteria that may be present on the knob 22. As shown in FIG. 7, the used towelette 10 is discarded into a receptacle 24. Preferably, the used towelette 10 is discarded without making any contact with the receptacle.

Having described the invention in detail, it is to be understood that this description is for illustrative purposes only. The scope and breadth of the invention shall be limited only by the appended claims.

I claim:

1. A method of washing hands with a towelette, the method comprising the steps of:
imprinting a message upon the towelette;
impregnating the towelette with a cleaning agent;
concealing the message;
rubbing a selected area of skin with the towelette, thereby cleansing the selected area and enabling the message to become apparent.

2. The method as in claim 1, further comprising the steps of running water over the selected area.

3. The method as in claim 2, further comprising the step of
performing the rubbing step adjacent a water faucet having at least one knob;
positioning the towelette over the at least one knob;
turning the at least one knob to cease flow of the running water.

4. The method as in claim 1, wherein the imprinting step further includes the step of
using a substantially clear oleophilic compound to draft the message upon the towelette.

5. The method as in claim 4, wherein the oleophilic compound is one of a terpene or a sterol.

6. The method as in claim 4, wherein the oleophilic compound comprises camphor oil.

7. The method as in claim 4, the concealing step including the step of applying a powdery soap to the towelette, thereby covering the message.

8. The method as in claim 1, the impregnating step further including the step of
moistening the towelette; and,
sprinkling a powdery soap over the towelette.

9. The method as in claim 7, further comprising the step of drying the towelette.

10. The method as in claim 1, wherein the cleaning agent includes a cholorhexidine alcohol solution.

11. The method as in claim 1, wherein the cleaning agent includes antimicrobial soap.

12. A towelette, comprising:
a porous cloth-like material;
a cleaning agent impregnating the material;
a message concealed within the material; wherein, as one rubs a selected area of skin with the material, the area is cleansed and the message converts from concealed to visible.

13. The towelette as in claim 12, further comprising a
substantially clear oleophilic compound that creates the message upon the towelette; wherein,
as the area is cleansed, the oleophilic compound gathers dirt, oils, and grime, thereby revealing the message.

14. The towelette as in claim 13, wherein the oleophilic compound is one of a terpene or a sterol.

15. The towelette as in claim 13, wherein the oleophilic compound comprises camphor oil.

16. The towelette as in claim 12, wherein the cleaning agent includes a powdery soap.

17. The towelette as in claim 16, wherein the cleaning agent includes an opaquing material that at least partly conceals the message, and wherein, as the area is cleansed, the cleaning agent erodes from the material and reveals the message.

18. The towelette as in claim 12, wherein the message is imprinted in a central location on the towelette, and the cleaning agent includes a generally opaque soap that is given greater concentration in the generally central location, thereby substantially obscuring the message until the cleaning agent is removed by a washing procedure using the towellete.

19. The towelette as in claim 12, wherein the message is imprinted using a solution that alters its color when wet.

20. A sanitation method comprising the steps of:
providing a towelette;
using a substantially clear oleophilic compound to imprint a message upon the towelette;
impregnating the towelette with a cleaning agent that includes at least one of a powdery soap, an antibacterial soap, or cholorhexidine alcohol solution;
running water over a selected area of skin;
rubbing the selected area of skin with the towelette while near a water faucet having at least one knob; thereby cleansing the selected area and enabling the message to become apparent;
performing the rubbing step adjacent a water faucet having at least one knob;
positioning the towelette over the at least one knob;
turning the at least one knob to cease flow of the running water; and,
discarding the towelette in a receptacle without making contact with the receptacle.
